(19) United States
(12) Patent Application Publication
(10) Pub. No.: US 2012/0296194 A1
(43) Pub. Date: Nov. 22, 2012

(54) METHOD AND SYSTEM FOR DETERMINING A CONDITION OF A BRAIN

(75) Inventors: SZE LOK SHERLOCK AU, NEW TERRITORIES (HK); SHENG JIN, NEW SOUTH WALES (AU)

(73) Assignee: GLOBAL ADVANCED VISION LIMITED, KWAI FONG (HK)

(21) Appl. No.: 13/472,385

(22) Filed: May 15, 2012

(30) Foreign Application Priority Data
May 16, 2011 (HK) 11104766.1

(51) Int. Cl. A61B 5/055

(52) U.S. Cl. 600/410

(57) ABSTRACT

A system for use in determining a condition of a brain, the system including an input device for providing data representing a structure of the brain and a processor communicably connected to the input device. The processor is configured for measuring at east one geometric characteristic of the structure of the brain by reference to the data, determining a brain index value by reference to the at least one geometric characteristic, and, comparing the brain index value against at least one predetermined index whereby, based on a result of the comparison, a condition of the brain is able to be determined corresponding to the brain index value.

### TECHNICAL FIELD

The present invention relates to a method and system for determining a condition of a brain and in particular, a disease, age or cognitive function of the brain.

### BACKGROUND OF THE INVENTION

The healthiness of a person's brain is typically determined by subjecting the person to a series of behavioral tests and exercises relating to memory, coordination, orientation, speech and the like, and observing and interpreting the results of the tests and exercises.

Unfortunately, discrepancies in the interpretation of the results tend to arise due to differences in the levels of skill and understanding of each tester, as well as the subjective nature of the interpretation performed by each tester. Accordingly, there is a perception that existing approaches for assessing the condition of a person's brain is susceptible to inconsistency and inaccuracy.

### SUMMARY OF THE INVENTION

The present invention seeks to alleviate at least one of the above-described problems.

The present invention may involve several broad forms. Embodiments of the present invention may include one or any combination of the different broad forms herein described.

In a first broad form, the present invention provides a system for use in determining a condition of a brain, the system including:

an input device for providing data representing a structure of the brain;

a processor communicably connected to the input device, the processor being configured for:

(i) measuring at least one geometric characteristic of the structure of the brain by reference to the data;

(ii) determining a brain index value by reference to the at least one geometric characteristic; and

(iii) comparing the brain index value against at least one predetermined index whereby, based on a result of the comparison, a condition of the brain is able to be determined corresponding to the brain index value.

Preferably, the data may include an image data representing an image of the structure of the brain. Preferably, the input device may include an image scanner for scanning the image data representing the structure of the brain. More preferably, the image scanner may include a magnetic image resonance scanner.

Typically, the structure of the brain may include at least one of a cortical sulcal structure of the brain and an outer cortex of the brain. Typically, the cortical sulcal structure may include one or more cortical sulci of the brain.

Typically, the cortical sulcal structure may include at least one of a superior frontal sulcus in a frontal lobe of the brain, an intra-parietal sulcus in the parietal lobe, a superior temporal sulcus in the temporal lobe, a cingulate sulcus in a limbic area of the brain, an interlobal sulcus of a central sulcus, and, an interlobal sulcus of a Sylvian fissure.

Typically, the geometric characteristic of the structure of the brain may include at least one of a shape, depth, width, length, curvature, area and a volume of the structure of the brain.

Typically, the brain index value may include a ratio of a surface area of the cortical sulcal structure against a surface area of the outer cortex of the brain. Advantageously, the brain index value does not take into account the gray matter intensity and is therefore not sensitive to its possible variations which have been problematic in conventional devices and processes for determining brain conditions.

Typically, the brain index value may include a span of the cortical sulcal structure of the brain. More typically, the span of the cortical sulcal structure may include an average distance between opposing gyral banks of the cortical sulcal structure.

Preferably, the present invention may be configured for determining a brain index value in respect of a hemisphere of the brain. Typically, the present invention may determine a brain index value of each hemisphere of the brain separately where the resulting brain index values in respect of each hemisphere of the brain may be used independently or collectively in determining a condition of the brain.

Typically, the condition of the brain may be indicative of an absence, presence, and/or severity of a disease affecting the brain. Also typically, the condition of the brain may be indicative of an age of the brain. More typically, the condition of the brain may be indicative of a cognitive function of the brain.

Preferably, the processor may be configured to compare the brain index value against a plurality of predetermined indices whereby one or more conditions of the brain are able to be determined.

Preferably, the processor may be configured for comparing the brain index value against at least one historical brain index value to determine an amount or rate of change in the condition of the brain.

Preferably, the present invention may include a memory store for storing brain index values determined by the processor. Typically, the memory store may include a database.

Typically, the brain index values stored in the memory store may be used as historical brain index values for comparison to determine an amount or rate of change in a condition of the brain.

Typically, the at least one predetermined index is able to be adjusted with reference to the result of comparison between the determined brain index value and the at least one predetermined index. Advantageously, this may assist in improving an accuracy and a value of the at least one predetermined index.

In a second broad form, the present invention provides a method for use in determining a condition of a brain, the method being operable using a computerised system including:

an input device
a processor communicably connected to the input device via a communication link; and
an output device communicably connected to the processor via the communication link;
the method including steps of:

(i) providing via the input device, data representing a structure of the brain;

(ii) the processor:
(a) measuring a least one geometric characteristic of the structure of the brain by reference to the data;
(b) determining a brain index value by reference to the at least one geometric characteristic; and
(c) comparing the brain index value against at least one predetermined index whereby, based on a result of the comparison, a condition of the brain is able to be determined corresponding to the brain index value; and

(iii) outputting the condition of the brain via the output device.

Preferably, the data may include an image data representing an image of the structure of the brain. Also preferably, the input device may include an image scanner for scanning the image data representing the structure of the brain, More preferably, the image scanner may include a magnetic image resonance scanner.

Typically, the structure of the brain includes at least one of a cortical sulcal structure of the brain and an outer cortex of the brain. Also typically, the cortical sulcal structure may include one or more cortical sulci of the brain.

Typically, the cortical sulcal structure may include at least one of a superior frontal sulcus in a frontal lobe of the brain, an intra-parietal sulcus in the parietal lobe, a superior temporal sulcus in the temporal lobe, a cingulate sulcus in a limbic area of the brain, an interlobal sulcus of a central sulcus, and, an interlobal sulcus of a Sylvian fissure.

Typically, the geometric characteristic of the structure of the brain may include at least one of a shape, depth, width, length, curvature, area and a volume of the structure of the brain.

Typically, the brain index value may include a ratio of a surface area of the cortical sulcal structure against a surface area of the outer cortex of the brain. Typically, the present invention may determine a brain index value of each hemisphere of the brain separately where the resulting brain index values in respect of each hemisphere of the brain may be used independently or collectively in determining a condition of the brain.

Typically, the brain index value may include a span of the cortical sulcal structure of the brain. Also typically, the span of the cortical sulcal structure may include an average distance between opposing gyral banks of the cortical sulcal structure.

Typically, the brain index value is determined in respect to a hemisphere of the brain.

Typically, the condition of the brain may be indicative of an absence, presence, and/or severity of a disease affecting the brain. Also typically, the condition of the brain may be indicative of an age of the brain. More typically, the condition of the brain may be indicative of a cognitive function of the brain.

Preferably, the present invention may include a step of comparing the brain index value against a plurality of predetermined indices whereby one or more conditions of the brain are able to be determined.

Preferably, the present invention may include a step of comparing the brain index value against at least one historical brain index value to determine an amount or rate of change in the condition of the brain.

Preferably, the present invention may include a step of storing brain index values determined by the processor in a memory store. Typically, the memory store may include a database.

Preferably, the present invention may include a step of using the brain index values stored in the memory store as historical brain index values for the comparison between the brain index value and the at least one historical brain index value to determine an amount or rake of change in a brain condition.

Typically, the present invention includes a step of adjusting the at least one predetermined index with reference to the result of the comparison between the determined brain index value and the at least one predetermined index.

In a third broad form, the present invention provides a data indicative of a brain condition, said data being produced in accordance with the method of the second broad form of the present invention or using the system of the first broad form of the present invention.

### BRIEF DESCRIPTION OF THE DRAWINGS

The present invention will become more fully understood from the following detailed description of a preferred but non-limiting embodiment thereof, described in connection with the accompanying drawings, wherein:

FIG. 1 shows a first embodiment computerised system for determining a condition of a brain;

FIG. 2 shows a flowchart of second embodiment method steps for using the computerised system of FIG. 1.

FIG. 3 shows an exemplary MRI image of a brain with both brain-tissue and non-brain tissue prior to pre-processing of the image;

FIGS. 4(a) and 4(b) show exemplary pre-processed MRI images of the brain in which non-brain-tissue has been removed and the boundaries between the remaining brain-tissue is highlighted;

FIGS. 5(a) and 5(b) show exemplary views of an MRI image of the extracted hemispheric white matter mesh (a) and gray matter mesh (b);

FIGS. 6(a) depicts an image exemplifying a brain having a relatively high degree of folding of the cerebral cortex;

FIGS. 6(b) depicts an image exemplifying a brain having a relatively low degree of folding of the cerebral cortex; and

FIG. 7 shows exemplary view of the sulcal span of a sulcal structure.

### DETAILED DESCRIPTION OF PREFERRED EMBODIMENTS

Preferred embodiments of the present invention will now be described below with reference to the accompanying drawings.

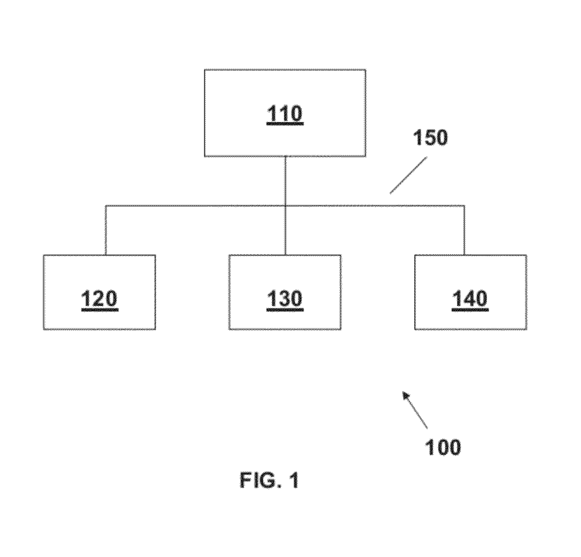

FIG. 1 depicts a schematic block diagram of a computerised system (100) for determining a condition of a brain. The condition of the brain could be indicative of an absence, presence and/or severity of a brain-related disease, it could be indicative of an age of the brain, or it could be indicative of a cognitive function of the brain.

In the preferred embodiments, the computerised system (100) includes a microprocessor (110) which is communicably connected via a communication link (150) to an input image scanner (120) such as a magnetic image resonance scanner, an output image display (130) such as an liquid crystal display, and a memory store (140) including a database.

The communication link (150) could include any suitable link wired or wireless link via which data can be electronically communicated between the components of the computerised system, The communication link (150) could for instance include data bus lines, a USB cable, a local area network, a wide area network, and the Internet. The computerised system (100) could include an integrated device such as a portable unit. Alternatively, the computerised system (100) could be implemented as a distributed system whereby its component units are remotely connected via the communication link (150). By way of example, the input image scanner (130) could be located at a medical clinic where an image of a patient's brain is scanned. The microprocessor (110) could be located at a remote premises and be configured for receiving the image data for processing via the Internet and a front-end web site interfaced with the microprocessor (110).

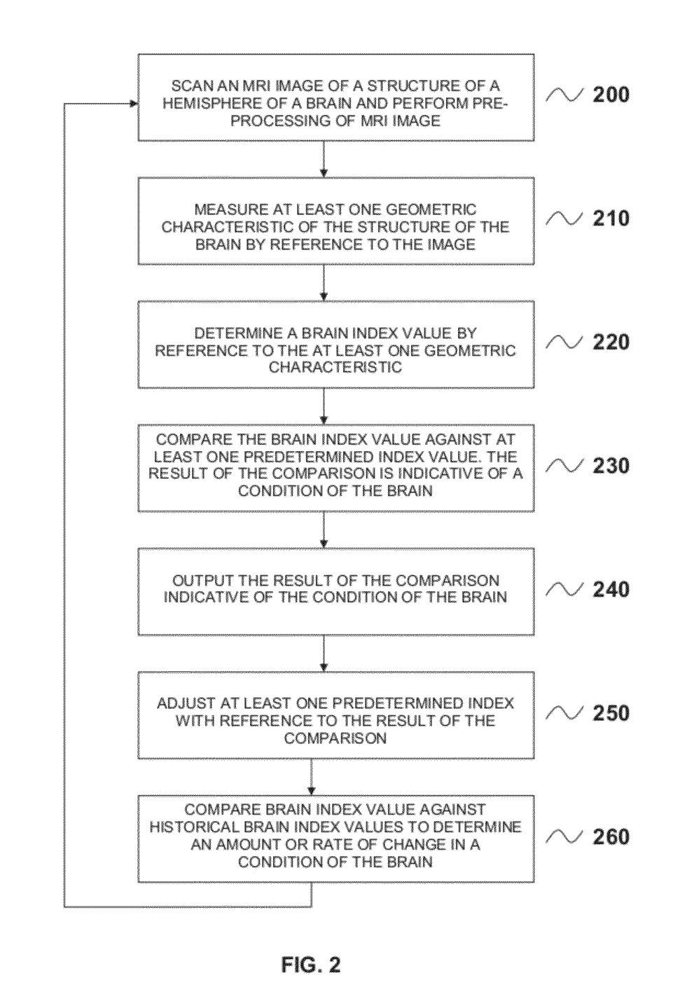

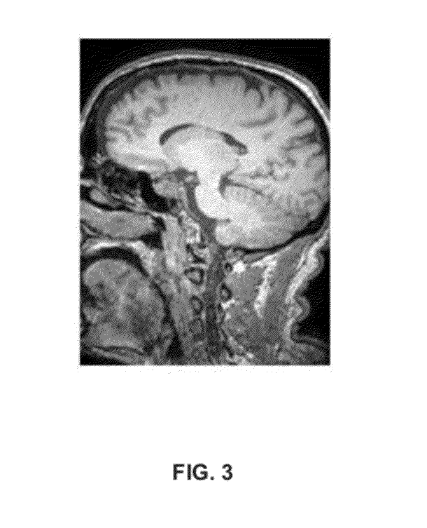

FIG. 3 shows an image of a brain captured by the MRI scanner (120). Pre-processing of the MRI image is performed in order to allow suitable identification and analysis of regions of the brain structure which can be used in the preferred embodiments to calculate a brain index value. The brain index value is thereafter able to be compared against various predetermined indices, with the results of the comparisons providing an indication of one or more condition(s) of the brain. In embodiments of the invention described herein, separate brain index values are calculated in respect of each of the hemispheres of the brain. The step of capturing and pre-processing the MRI image is represented by block 200 in FIG. 2.

Pre-processing of the MRI image is performed by the microprocessor (110) using any suitable image processing software such as Brainvisa or SPM5. Regions of the MRI image representing non-brain tissue such as skull, muscle fat and the like are removed no enable ease of identification and analysis of the brain-related tissue including grey matter (GM), white matter (VVM) and cerebrospinal fluid (CSF).

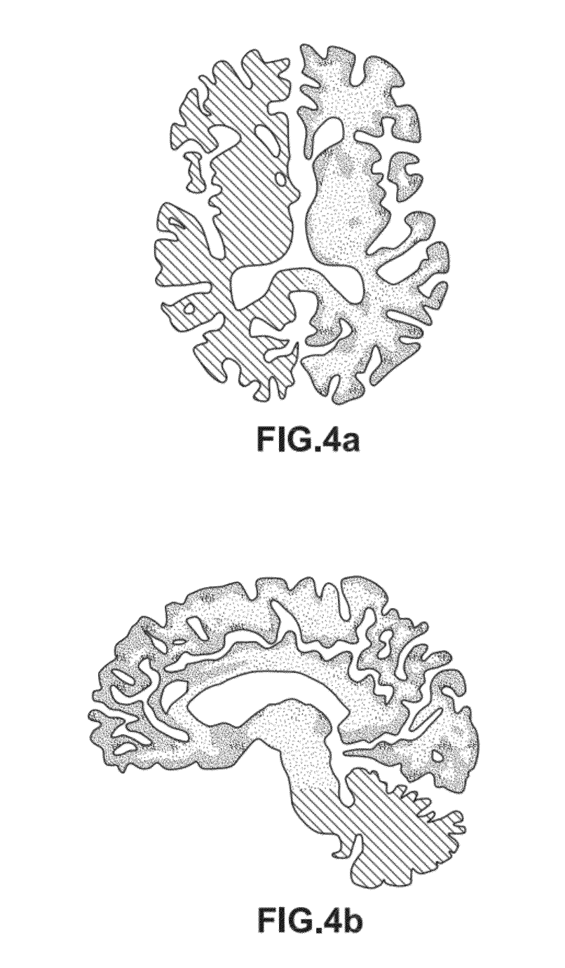

The microprocessor (110) is also configured to apply a fuzzy-classifier-based anatomical segmentation algorithm to further segment the brain tissue into GM. WM and CSF regions after applying a field inhomogeniety bias correction algorithm. FIGS. 4(a) and 4(b) show an image of the brain highlighting the boundaries between the GM, WM and the CSF.

The microprocessor (110) is further configured to process the image shown in

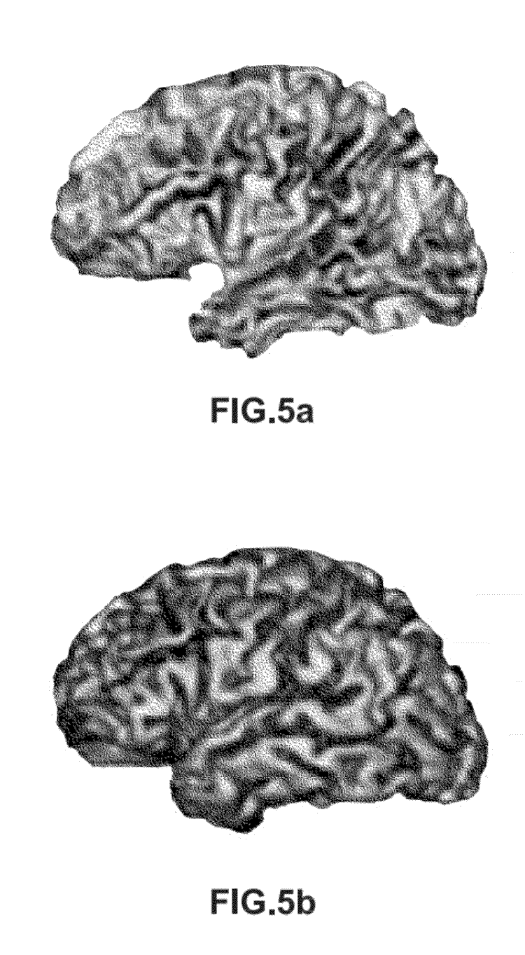

FIGS. 4(a) and 4(b) in order to extract hemispheric meshes by reference to the highlighted boundaries between GM. WM and CSF, FIGS. 5(a) and 5(b) show an image of the extracted hemispheric white (a)/gray matter (b) meshes.

Thereafter, the medial surface of the cortical folds is calculated by the microprocessor (110) by applying a homotopic erosion algorithm. A crevasse detector reconstructs sulcal structures as medial surfaces from the two opposing gyral banks that span from the most internal point of sulcal fold to the convex hull of the cortex. Spatial normalisation could be applied to the image data if desired.

Thereafter, the microprocessor (110) is configured for identifying the different cortical sulcal structures in each hemisphere of the brain by applying a pattern recognition algorithm. A cortical sulcal is a depression or fissure in the outermost surface of the brain known as the cerebral cortex, Each hemisphere of the brain includes several different cortical sulci of varying configurations and dimensions which form part of the cerebral cortex, Each cortical sulcus of the brain is each surrounded on each side by opposing gyral walls.

The pattern recognition can be performed by suitable software such as

Brainvisa executable by the microprocessor (110). Although it is possible for the microprocessor (110) to identify a relatively large number of cortical sulcal structures in each hemisphere of the brain, in the preferred embodiments, the sulcal structures identified and labeled include at least the superior frontal sulcus in the frontal lobe, the intra-parietal sulcus in the parietal lobe, the superior temporal sulcus in the temporal lobe, the cingulate sulcus in the limbic area, the interlobal sulcus of the central sulcus, and, the interlobal sulcus of the Sylvian fissure. These sulcal structures are considered particularly useful as they are present in brain structures of all human subjects, they are located in different cerebral lobes of the brain, and, are they relatively large which assists provides greater ease of identification.

Once the sulcal structures of the brain are suitably identified and labeled, the microprocessor (110) is configured for measuring geometric characteristics of the sulcal structures such as shape, depth, width, length, curvature, area and a volume of the cortical sulci (either individually or collectively) and the outer cortex of the brain. The geometric characteristics are able to be used to determine a brain index value which can be compared against various predetermined indices to provide indications of the absence, presence, and/or severity of various diseases, an age or cognitive function of the brain. The step of measuring the geometric characteristics of the brain structure is represented by block 210 in FIG. 2.

In the preferred embodiments, the brain index value is calculated based on geometric characteristics of the brain structure identifiable on each hemisphere of the brain. The brain index value determined in respect of each hemisphere of the brain can be used independently for comparison with predetermined indices to provide an indication of the absence, presence and/or severity of a brain-related disease, cognitive function or an age of the brain, or, collectively to provide such an indication. The step of calculating a brain index value is represented by block 220 in FIG. 2.

The brain index value can be determined in at least two ways by the microprocessor (110). The first brain index value, referred to as a global sulcal index (GSI), is determined based on a ratio of the total sulcal area of the brain against the outer cortex area of the hemisphere of the brain. The total sulcal area is calculated as the sum of the area of all the segmented cortical folds, with the area of each cortical fold being defined as the sum of all the triangle areas defining the fold mesh. The outer cortex area of the brain structure is defined as the area of a smooth envelope of the brain mask. The envelope is created by morphologic closing of the brain mask using imaging processing techniques.

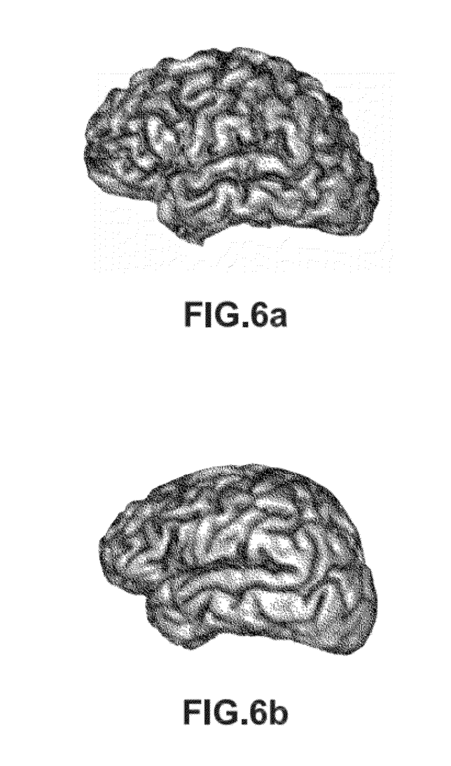

In respect to calculation of this first brain index value, it would be appreciated that a cortex with extensive folding such as shown in FIG. 6(a) will have a relatively large GSI value whereas a cortex with a low degree of folding such as shown in FIG. 6(b) will have a relatively small GSI value. If the cortex area is maintained constant, the GSI will increase with an increase in the number and/or surface area of sulcal folds, and a diminishing of convolutional patterns in the cerebral cortex will result in the GSI value converging to zero.

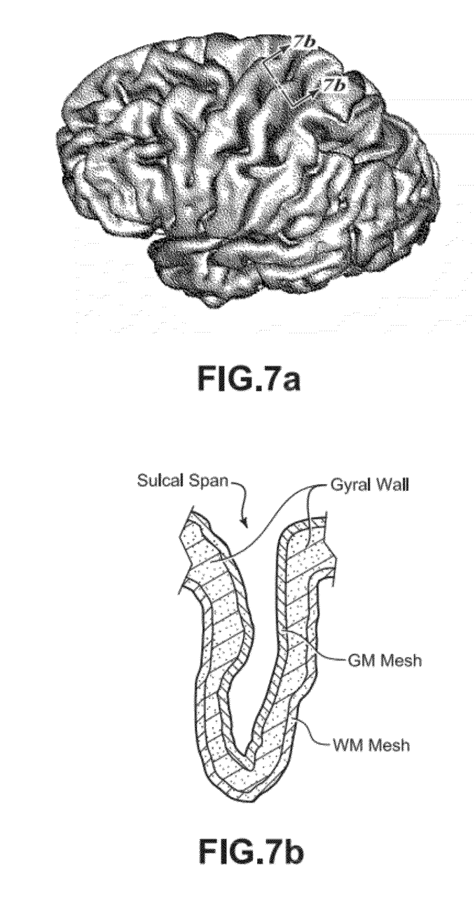

An additional and/or alternative brain index value can also be calculated based upon the average sulcal span for an individual sulcal structure. In the preferred embodiments, the sulcal span is defined as an average three-dimensional distance between opposing gyral banks of the sulcal along the normal projections to the medial sulcal mesh as depicted in FIG. 7. The high resolution medial sulcal meshes for each sulcus are created using Brainvisa software in this embodiment. Geometrically, a medial sulcal mesh traverses the sulcal space in the middle of the sulcal span, parallel to the gyral GM borders and spans the entire sulcal depth. To calculate the sulcal span of a given sulcus, the sulcus is identified by using the medial sulcal mesh for each sulcus to seed a three dimensional region growing algorithm so as to mask the GSF volume with this sulcal structure. To prevent the region-growing from spilling over into intersecting sulci, points that are more than 10 mm away from the sulcal skeleton are not considered by the algorithm. The convex hull of the cortical GM ribbon is then used to isolate sulcal space from the subarachnoid GSF. The Euclidean distance between two points residing on the gyral mesh on either side of the sulcal surface is then taken as the sulcal span.

The microprocessor (110) is further configured for comparing the calculated brain index value (the GSI value and/or the sulcal span) against predetermined indices. This step is represented by block 230 in FIG. 2. The predetermined indices are arranged against various parameters including the subject age, sex and the particular hemisphere of the brain used in calculation of the brain index. The predetermined indices provide pre-compiled associations between the respective brain index values and indications of the absence, presence and/or severity of a given brain condition (e.g. age, cognitive function, and brain-related disease). Accordingly, when a given brain index value is compared against the predetermined indices it is possible to obtain an objective indication of the condition of the brain being tested.

GSI brain index values tend to decline faster for men than with women due to age, Sulcal span brain index values also tend to increase more significantly for men than women with ago. Accordingly, these parameters should be considered in the process of pre-compiling the predetermined indices which are used in comparison with calculated brain index values. Moreover, the choice of predetermined indices used in the comparison with a calculated brain index value should be suitably selected based on parameters of the subject tested such as sex, age, hemisphere of the brain and so on. The microprocessor (110) could be configured to automatically compare appropriate predetermined indices against the calculated brain index value based on the parameters such as age, sex and the like entered into the microprocessor in respect to the specific subject being tested.

Upon comparison of the brain index value with appropriate predetermined indices matching the parameters of the subject tested, a result can be output upon the LCD (130) based on the comparison indicative of the brain condition. The step of outputting the indication of the brain condition via the LCD is represented by block 240 in FIG. 2.

The predetermined indices are stored in a database of the memory store (140) accessible by the microprocessor (110) via the communication link (150). In certain embodiments of the present invention, a predetermined index in the database could be updated and adjusted by reference to the result of each new comparison between a calculated brain index value and the predetermined index. In this manner, the perceived accuracy and value of any given predetermined index could be enhanced as the number of updates and adjustments are made based on each new comparison that is loaded into the database of predetermined indices, The step of adjusting the predetermined indices is represented by block (250) in FIG. 2.

The microprocessor (110) is also configured to compare the brain index value against one or more historical brain index values to provide an indication of the amount or rate of deterioration in a specific condition of the brain for a given subject being tested. Historical brain index values are also stored in the database (140). The historical brain index values can be updated each time a new comparison is made between the calculated brain index value and predetermined indices for a given subject. The step of comparing the brain index value against historical brain index values is represented by block 260 in FIG. 2.

Those skilled in the ark will appreciate that the invention described herein is susceptible to variations and modifications other than those specifically described without departing from the scope of the invention. All such variations and modification which become apparent to persons skilled in the art, should be considered to fall within the spirit and scope of the invention as broadly hereinbefore described. It is to be understood that the invention includes all such variations and modifications. The invention also includes all of the steps and features, referred or indicated in the specification, individually or collectively, and any and all combinations of any two or more of said steps or features.

The reference to any prior art in this specification is not, and should not be taken as, an acknowledgment or any form of suggestion that that prior art forms part of the common general knowledge.

1. A system for use in determining a condition of a brain, the system including:
an input device for providing data representing a structure of the brain;
a processor communicably connected to the input device, the processor being configured for:
(i) measuring at least one geometric characteristic of the structure of the brain by reference to the data;
(ii) determining a brain index value by reference to the at least one geometric characteristic; and
(iii) comparing the brain index value against at least one predetermined index whereby, based on a result of the comparison, a condition of the brain is able to be determined corresponding to the brain index value.

2. A system as claimed in claim 1 wherein the data includes an image data representing an image of the structure of the brain.

3. A system as claimed in claim 2 wherein the input device includes an image scanner for scanning the image data representing the structure of the brain.

4. A system as claimed in claim 3 wherein the image scanner includes a magnetic image resonance scanner.

5. A system as claimed in claim 1 wherein the structure of the brain includes at least one of a cortical sulcal structure of the brain and an outer cortex of the brain.

6. A system as claimed in claim 5 wherein the cortical sulcal structure includes one or more cortical sulci of the brain.

7. A system as claimed in claim 5 or 6 wherein the cortical sulcal structure includes at least one of a superior frontal sulcus in a frontal lobe of the brain, an intra-parietal sulcus in the parietal lobe, a superior temporal sulcus in the temporal lobe, a cingulate sulcus in a limbic area of the brain, an interlobal sulcus of a central sulcus, and, an interlobal sulcus of a Sylvian fissure.

8. A system as claimed in claim 1 wherein the geometric characteristic of the structure of the brain includes at least one of a shape, depth, width, length, curvature, area and a volume of the structure of the brain.

9. A system as claimed in claim 5 wherein the brain index value includes a ratio of a surface area of the cortical sulcal structure against a surface area of the outer cortex of the brain.

10. A system as claimed in claim 5 wherein the brain index value includes a span of the cortical sulcal structure of the brain.

11. A system as claimed in claim 10 wherein the span includes an average distance between opposing gyral banks of the cortical sulcal structure.

12. A system as claimed in claim 1 configured for determining a brain index value in respect of a hemisphere of the brain.

13. A system as claimed in claim 1 wherein the condition of the brain is indicative of an absence, presence, and/or severity of a disease affecting the brain.

14. A system as claimed in claim 1 wherein the condition of the brain is indicative of an age of the brain.

15. A system as claimed in claim 1 wherein the condition of the brain is indicative of a cognitive function of the brain.

16. A system as claimed in claim 1 wherein the processor is configured to compare the brain index value against a plurality of predetermined indices whereby one or more conditions of the brain are able to he determined.

17. A system as claimed in claim 1 wherein the processor is configured for comparing the brain index value against at least one historical brain index value to determine an amount or rate of change in the condition of the brain.

18. A system as claimed in claim 1 including a memory store for storing brain index values determined by the processor.

19. A system as claimed in claim 18 wherein the memory store includes a database.

20. A system as claimed in claim 18 or 19 wherein the brain index values stored in the memory store are able to be used as historical brain index values for comparison in claim 17.

21. A system as claimed in claim 1 wherein the at least one predetermined index is able to be adjusted with reference to the result of the comparison between the determined brain index value and the at least one predetermined index.

22. A method for use in determining a condition of a brain, the method being operable using a computerised system including:
an input device
a processor communicably connected to the input device via a communication link; and
an output device communicably connected to the processor via the communication link;
the method including steps of:
(i) providing via the input device, data representing a structure of the brain;
(ii) the processor:
(a) measuring a least one geometric characteristic of the structure of the brain by reference to the data;
(b) determining a brain index value by reference to the at least one geometric characteristic; and
(c) comparing the brain index value against at least one predetermined index whereby, based on a result of the comparison, a condition of the brain is able to be determined corresponding to the brain index value; and
(iii) outputting the condition of the brain via the output device.

23. A method as claimed in claim 22 wherein the data includes an image data representing an image of the structure of the brain.

24. A method as claimed in claim 23 wherein the input device includes an image scanner for scanning the image data representing the structure of the brain.

25. A method as claimed in claim 24 wherein the image scanner includes a magnetic image resonance scanner.

26. A method as claimed in claim 22 wherein the structure of the brain includes at least one of a cortical sulcal structure of the brain and an outer cortex of the brain.

27. A method as claimed in claim 26 wherein the cortical sulcal structure includes one or more cortical sulci of the brain.

28. A method as claimed in claim 26 or 27 wherein the cortical sulcal structure includes at least one of a superior frontal sulcus in a frontal lobe of the brain, an intra-parietal sulcus in the parietal lobe, a superior temporal sulcus in the temporal lobe, a cingulate sulcus in a limbic area of the brain, an interlobal sulcus of a central sulcus, and, an interlobal sulcus of a Sylvian fissure.

29. A method as claimed in claim 22 wherein the geometric characteristic of the structure of the brain includes at least one of a shape, depth, width, length, curvature, area and a volume of the structure of the brain.

30. A method as claimed in claim 26 wherein the brain index value includes a ratio of a surface area of the cortical sulcal structure against a surface area of the outer cortex of the brain.

31. A method as claimed in claim 26 wherein the brain index value includes a span of the cortical sulcal structure of the brain.

32. A method as claimed in claim 31 wherein the span includes an average distance between opposing gyral banks of the cortical sulcal structure.

33. A method as claimed in claim 22 wherein the brain index value is determined in respect of a hemisphere of the brain.

34. A method as claimed in claim 22 wherein the condition of the brain is indicative of an absence, presence, and/or severity of a disease affecting the brain.

35. A method as claimed in claim 22 wherein the condition of the brain is indicative of an age of the brain.

36. A method as claimed in claim 22 wherein the condition of the brain is indicative of a cognitive function of the brain.

37. A method as claimed in claim 22 including a step of comparing the brain index value against a plurality of predetermined indices whereby one or more conditions of the brain are able to be determined.

38. A method as claimed in claim 22 including a step of comparing the brain index value against at least one historical brain index value to determine an amount or rate of change in the condition of the brain.

39. A method as claimed in claim 22 including a step of storing brain index values determined by the processor in a memory store.

40. A method as claimed in claim 39 wherein the memory store includes a database.

41. A method as claimed in claim 39 or 40 including a step of using the brain index values stored in the memory store as historical brain index values for the comparison in claim 38.

42. A method as claimed in claim 22 including a step of adjusting the at least one predetermined index with reference to the result of the comparison between the determined brain index value and the at least one predetermined index.

43. A data indicative of a brain condition, said data being producing using the system of claim 1.

44. A data indicative of a brain condition, said data being produced in accordance with the method of claim 22.
